(19) United States
(12) Patent Application Publication
(10) Pub. No.: US 2026/0232647 A1
(43) Pub. Date: Aug. 13, 2026

(54) STABLE COMPOSITIONS OF BUPIVACAINE HYDROCHLORIDE

(71) Applicant: Nevakar Injectables Inc., Bridgewater, NJ (US)

(72) Inventors: Victoria Percoco, Old Bridge, NJ (US); Tushar Hingorani, Bridgewater, NJ (US); Kumaresh Soppimath, Skillman, NJ (US)

(73) Assignee: Nevakar Injectables Inc., Bridgewater, NJ (US)

(21) Appl. No.: 19/460,222

(22) Filed: Jan. 26, 2026

Related U.S. Application Data
(60) Provisional application No. 63/755,696, filed on Feb. 7, 2025.

(51) Int. Cl. A61K 31/445; A61K 9/00; A61K 9/08; A61K 47/02; A61K 47/12

(52) U.S. Cl. CPC A61K 31/445; A61K 9/0019; A61K 9/08; A61K 47/02; A61K 47/12

(57) ABSTRACT

Disclosed herein are stable injectable compositions of bupivacaine hydrochloride suitable for use in pre-filled syringes. Also disclosed herein are methods of preparing and using the same.

### TECHNICAL FIELD

The present disclosure pertains to stable compositions of bupivacaine hydrochloride, and particularly to stable injectable compositions of bupivacaine hydrochloride which are suitable for use in pre-filled syringes.

### BACKGROUND

The background description includes information that may be useful in understanding the present invention. It is not an admission that any of the information provided herein is prior art or relevant to the presently claimed invention, or that any publication specifically or implicitly referenced is prior art.

All publications and patent applications herein are incorporated by reference to the same extent as if each individual publication or patent application were specifically and individually indicated to be incorporated by reference. Where a definition or use of a term in an incorporated reference is inconsistent or contrary to the definition of that term provided herein, the definition of that term provided herein applies and the definition of that term in the reference does not apply.

Bupivacaine hydrochloride is a local anesthetic widely used in both superficial and invasive procedures. It is indicated, either alone or in combination with epinephrine, for producing local or regional anesthesia or analgesia during surgeries, dental and oral surgery procedures, diagnostic and therapeutic procedures, as well as obstetrical procedures. Bupivacaine hydrochloride has been sold for many years in the United States and elsewhere under the tradename Marcaine™ (Bupivacaine Hydrochloride Injection, USP, Hospira, Lake Forest, IL). Marcaine™ is supplied as a sterile isotonic solution in single-dose and multi-dose glass vials, with or without epinephrine, for injection via local infiltration, peripheral nerve block, and caudal or lumbar epidural blocks. Each mL of Marcaine™ contains 2.5 mg (0.25%), 5 mg (0.5%), or 7.5 mg (0.75%) of bupivacaine hydrochloride, along with sodium chloride as tonicity agent, and sodium hydroxide and hydrochloric acid as pH adjusters in water for injection. The pH of Marcaine™ is between 4.0 and 6.5. This prior art composition is referred to throughout this disclosure as the “currently available bupivacaine composition”. While this composition has demonstrated reasonable stability in glass vials and has been successfully marketed in this form for many years, it has been found to be unstable when stored in pre-filled syringes. In particular, the composition exhibits a lack of pH stability in pre-filled syringes, with a downward drift of up to 2 pH units. This pH decrease significantly impacts the stability of the composition over its shelf life and leads to a marked acidification upon storage in pre-filled syringes. Administration of acidic drug product may lead to injection site irritation especially in the epidural space.

Another bupivacaine formulation is described in WO 2025/015011 where an unbuffered and preservative-free bupivacaine-containing solution is filled in certain multi-layer polymeric bags having a volume of at least 50 mL to so achieve better pH and/or product stability. Similarly, US 2025/0082622 teaches a bupivacaine-containing solution with improved pH stability during autoclaving. However, such formulations are generally used for administration of relatively large volumes. More concentrated forms of bupivacaine are described in U.S. Pat. No. 11,672,863. However, solubility is increasingly challenging and such formulations require addition of at least three different acids. In yet other approaches, stability of bupivacaine can be achieved by enclosing the active ingredient in microspheres as disclosed in US 2024/0033221 or in multivesicular liposomes as discussed in U.S. Pat. No. 11,426,348. While allowing for increased storage stability, encapsulation typically requires complex processes that will increase the risk of contamination and/or cost of manufacture.

Therefore, there is a need to develop new and improved injectable compositions of bupivacaine hydrochloride that remain stable when stored in pre-filled syringes and exhibit minimal or no pH drift over time.

### SUMMARY

The present disclosure provides stable injectable compositions of bupivacaine hydrochloride specifically designed for use in pre-filled syringes. The inventor has now discovered that buffering the currently available bupivacaine composition at a pH ranging from about 4.0 to about 6.5, using an appropriate buffer, ensures its stability in pre-filled syringes and effectively prevents or minimizes pH drift throughout the composition's shelf life. The composition disclosed herein demonstrates significantly improved stability in pre-filled syringes compared to the currently available bupivacaine composition and, more importantly, remains stable for a commercially viable period in pre-filled syringes. This enhanced stability enables the convenient use of bupivacaine hydrochloride in pre-filled syringe formats.

Accordingly, one aspect of the present disclosure is directed to a pharmaceutical composition in a pre-filled syringe, comprising: bupivacaine hydrochloride, a tonicity agent, a buffer, a pH adjuster in an amount sufficient to adjust the composition to an initial pH of about 4.0 to about 6.5, and water. The pharmaceutical compositions disclosed herein are characterized by exhibiting a pH drift of less than 0.5 pH unit after storage in pre-filled syringes for at least 2 months, preferably at least 6 months, more preferably at least 12 months, even more preferably at least 18 months, and most preferably at least 24 months, at room temperature (e.g., 15° C. to 25° C.). In certain embodiments, the compositions disclosed herein exhibit a pH drift of less than 0.5 pH unit after being stored for at least one week under accelerated storage conditions of 60° C. Suitable buffers for use in the present invention include citric acid, sodium citrate, lactic acid, sodium lactate, and combinations thereof. In some embodiments, the buffer used is citric acid. In some embodiments, the buffer used is lactic acid. In certain preferred embodiments, the buffer will be employed in the composition at a concentration of from about 0.05 mg/mL to about 10 mg/mL of the composition. Preferably, the initial pH of the composition is between 4.0 and 6.5 or between 4.5 and 6.3 or between 5.0 and 6.2. The pH of the composition is adjusted with a pH adjuster, which is preferably sodium hydroxide and/or hydrochloric acid. The pharmaceutical compositions described herein can have a bupivacaine hydrochloride concentration in the range of from about 1 mg/mL to about 10 mg/mL of the composition. In certain embodiments, the compositions include bupivacaine hydrochloride at a concentration of about 2.5 mg/mL (0.25%), about 5 mg/mL (0.5%), or about 7.5 mg/mL (0.75%) of the composition.

In some embodiments, the pharmaceutical composition described herein may further include a tonicity agent. The tonicity agent may be selected from a group consisting of sugar (such as dextrose), glycerin, a sugar alcohol (such as mannitol), sodium chloride, and combinations thereof. The concentration of the tonicity agent may be at a concentration of from about 5 mg/mL to about 15 mg/mL of the composition. Preferably, sodium chloride is used as the tonicity agent.

Another aspect of the present disclosure is directed to a method for improving stability of an injectable solution composition comprising about 1 mg/mL to about 10 mg/mL of bupivacaine hydrochloride, about 5 mg/mL to about 15 mg/mL of a tonicity agent, and water, with a pH ranging from about 4.0 to 6.5. The method involves incorporating a buffer into the composition such that the composition has a pH drift of less than 0.5 pH unit after storage for at least 2 months in a pre-filled syringe at 25° C. or after storage for at least one week in a pre-filled syringe under accelerated conditions of 60° C.

Another aspect of the present disclosure is directed to a method of preventing acidification of an injectable solution composition comprising about 1 mg/mL to about 10 mg/mL of bupivacaine hydrochloride, about 5 mg/mL to about 15 mg/mL of a tonicity agent and water. Preferably, the initial pH of the composition is between 4.0 and 6.5 or between 4.5 and 6.3 or between 5.0 and 6.2. The pH of the composition is adjusted with a pH adjuster, which is preferably sodium hydroxide and/or hydrochloric acid. In certain embodiments the initial pH is about 5.5 or about 6.0 or about 6.5. In certain embodiments, the composition disclosed herein exhibits a pH drift of less than 0.5 pH units or about 1 pH units or about 1.5 pH units when stored at room temperature (15° C. to 25° C.) for at least 6 months, more preferably 12 months, more preferably 18 months or most preferably about 24 months.

The present disclosure also provides a pre-filled syringe comprising the pharmaceutical composition disclosed herein.

The present disclosure further provides a kit comprising a pre-filled syringe containing the pharmaceutical composition disclosed herein, and instructions for using the pharmaceutical composition.

Various objects, features, aspects, and advantages of the inventive subject matter will become more apparent from the following detailed description of preferred embodiments.

### DETAILED DESCRIPTION

The following is a detailed description of embodiments of the present disclosure. The embodiments are in such detail as to clearly communicate the disclosure. However, the amount of detail offered is not intended to limit the anticipated variations of embodiments; on the contrary, the intention is to cover all modifications, equivalents, and alternatives falling within the spirit and scope of the present disclosure as defined by the appended claims.

Unless otherwise noted, all measurements are in standard metric units.

Unless otherwise noted, all instances of the words “a,” “an,” or “the” can refer to one or more than one of the word that they modify.

Unless otherwise noted, the phrase “at least one of” means one or more than one of an object.

The term “about” as used herein contemplates what is ordinarily meant in the art, and includes an acceptable range for a particular value as determined by a skilled artisan in the art. This, in turn, may depend on how the value is determined, or it may depend on the result to be achieved. For example, used in the context of chemical compositions, the term “about” allows for a degree of variation inherent in the methods utilized for measuring the chemical compounds, and/or also allows for a degree of variation that produces the same or similar result in term of desired effectiveness. In some embodiments, the term “about” when used before a numerical value indicates that the value may vary within reasonable range, such as ±5%, ±1%, and ±0.2%. When “about” is used before a pH value, it indicates that the pH value may vary ±0.2, ±0.1, ±0.05 or ±0.02 pH units.

Unless otherwise noted, when a range of numbers refers to numbers that are easily and routinely varied in a laboratory setting, such as weight, mass, concentration, temperature, or pressure, then the range of numbers or measurements includes all numbers in that range. For example, the range of 1 mg/mL to 10 mg/mL includes 2.0, 3.0, 4.0, 5.0, 6.0, 7.0, 8.0, 9.0 and any sub range/number therein.

Unless otherwise noted, the term “for example” or “e.g.,” as used herein, is used merely by way of example, and should not be construed as limiting the present disclosure to only those items explicitly referred to in the specification.

Unless otherwise noted, all reactions, procedures, and formulations were or can be performed under normal temperature and pressure.

The term “pH stable” means that the composition has a pH drift of less than 1.0 for an indicated length of time, under the stated stability conditions. A pH stable composition is contemplated to have a pH drift of less than 1.0, or more preferably pH drift of less than 0.9, or more preferably pH drift of less than 0.8, or more preferably pH drift of less than 0.7, or more preferably pH drift of less than 0.6, or more preferably pH drift of less than 0.5, or more preferably pH drift of less than 0.4, or more preferably pH drift of less than 0.3, or more preferably pH drift of less than 0.2, or most preferably pH drift of less than 0.1 for an indicated length of time, under the stated stability conditions.

As used herein, the term “Prefilled syringe” refers to a ready-to-use non-reusable product. This type of syringe is filled with a medication and stored in this pre-filled form for a period of time before administration of the medication to an individual.

Marcaine™, an FDA-approved formulation of bupivacaine hydrochloride marketed at concentrations of 2.5 mg/mL, 5 mg/mL, and 7.5 mg/mL in glass vials, exhibits a lack of pH stability in pre-filled syringes, resulting in a pH drift outside the FDA-recommended range of 4.0 to 6.5, with a downward drift of up to 2 pH units. The present disclosure provides stable injectable compositions of bupivacaine hydrochloride specifically designed for use in pre-filled syringes. The inventors of the present disclosure have unexpectedly discovered that incorporating a buffer designed to maintain a pH range of 4.0 to 6.5 into the currently available bupivacaine composition prevents or minimizes pH drift during the composition's shelf life and ensures its stability in pre-filled syringes. The compositions described herein demonstrate significantly improved stability in pre-filled syringes compared to the currently available bupivacaine composition and, more importantly, remain stable for a commercially viable period in pre-filled syringes. This enhanced stability facilitates the convenient sale of bupivacaine hydrochloride in pre-filled syringe formats.

In one embodiment, disclosed herein is a pharmaceutical composition in a pre-filled syringe, comprising: bupivacaine hydrochloride, a buffer, a pH adjuster in an amount sufficient to adjust the composition to an initial pH of about 4.0 to about 6.5, and water. The term “initial adjusted pH” or “initial pH” as used herein, refers to the pH level initially set during the preparation of the composition. Once the composition is prepared and its pH is adjusted to the initial value, it is expected that, when stored in pre-filled syringes, the composition will exhibit a pH drift of less than 0.5 pH unit for at least 2 months, preferably at least 6 months of storage at room temperature, or for at least one week of storage under accelerated conditions of 60° C.

In some embodiments, the initial adjusted pH of the composition is from about 4.5 to about 5.6. In some embodiments, the initial adjusted pH of the composition is from about 5.4 to about 6.5. In some embodiments, the initial adjusted pH of the composition is from about 6.0. In some embodiments, the initial pH of the composition is between 4.5 and 6.3, between 5.0 and 6.2, between 4.0 and 5.6, between 5.4 and 6.5, or 5.5.

Typically, the contemplated compositions will be available in a range of concentrations commonly required by medical practitioners for the anesthesia. In some embodiments, the pharmaceutical compositions described herein have a bupivacaine hydrochloride concentration in the range from about 1 mg/mL to about 10 mg/mL of the composition. In certain embodiments, the compositions include bupivacaine hydrochloride at a concentration of about 2.5 mg/mL (0.25%), about 5 mg/mL (0.5%), or about 7.5 mg/mL (0.75%) of the composition.

Suitable buffers for use in the pharmaceutical compositions described in the present disclosure are those with sufficient buffering capacity to maintain a pH range of 4.0 to 6.5. Exemplary buffers include, but are not limited to, citric acid, sodium citrate, lactic acid, sodium lactate, and combinations thereof. The buffer can be employed in the composition at a concentration of from about 0.05 mg/mL to about 10 mg/mL, from about 0.07 mg/mL to about 9 mg/mL, from about 0.09 mg/mL to about 8 mg/mL, from about 0.1 mg/mL to about 6 mg/mL, from about 0.15 mg/mL to about 5 mg/mL, from about 0.2 mg/mL to about 4 mg/mL, from about 0.2 mg/mL to about 3 mg/mL, from about 0.2 mg/mL to about 2 mg/mL, from about 0.5 mg/mL to about 6.5 mg/mL, from about 0.8 mg/mL to about 5.0 mg/mL, from about 0.9 mg/mL to about 4.0 mg/mL, from about 1.0 mg/mL to about 3.5 mg/mL, or from about 1.0 mg/mL to about 2.5 mg/mL of the composition.

A preferred embodiment of the present disclosure relates to a pharmaceutical composition wherein the buffer is citric acid present at a concentration of from about 0.05 mg/mL to about 5 mg/mL, from about 0.07 mg/mL to about 4 mg/mL, from about 0.09 mg/mL to about 4 mg/mL, from about 0.1 mg/mL to about 4 mg/mL, from about 0.15 mg/mL to about 3.5 mg/mL, from about 0.2 mg/mL to about 3 mg/mL, from about 0.2 mg/mL to about 2 mg/mL, or from about 0.2 mg/mL to about 1 mg/mL of the composition.

Another preferred embodiment of the present disclosure relates to a pharmaceutical composition wherein the buffer is lactic acid present at a concentration of from about 0.1 mg/ml to about 7 mg/mL, from about 0.5 mg/mL to about 6.5 mg/mL, from about 0.8 mg/mL to about 5.0 mg/mL, from about 0.9 mg/mL to about 4.0 mg/mL, from about 1.0 mg/mL to about 3.5 mg/mL, or from about 1.0 mg/mL to about 2.5 mg/mL of the composition.

The pH of the composition is adjusted with a pH adjuster, which is preferably sodium hydroxide, hydrochloric acid, or a combination thereof. In some embodiments, the pH adjuster is sodium hydroxide. In some embodiments, the pH adjuster is hydrochloric acid. In some embodiments, the pH adjuster is a combination of sodium hydroxide and hydrochloric acid. The concentration of the pH adjuster can be any concentration suitable for adjusting the composition to an initial pH of from about 4.0 to about 6.5. Preferably, the initial pH of the composition is between 4.5 and 6.3, between 5.0 and 6.2, between 4.0 and 5.6, between 5.4 and 6.5, or 5.5.

In one embodiment, the water used to prepare the pharmaceutical composition is of injection-grade quality. Water may be used in a quantity sufficient to make up the final volume of the composition.

The pharmaceutical composition described herein may further include a tonicity agent. The tonicity agent may be selected from group consisting of a sugar (such as dextrose), sodium chloride, potassium chloride and combinations thereof. The concentration of the tonicity agent may be at a concentration of from about 5 mg/mL to about 15 mg/mL, from about 5.5 mg/mL to about 12 mg/mL, from about 6.0 mg/mL to about 10 mg/mL, from about 6.5 mg/ml to about 10 mg/mL, from about 7.0 mg/mL to about 10 mg/mL, from about 7.0 mg/mL to about 9 mg/mL, or from about 7.0 mg/mL to about 8 mg/mL of the composition.

A preferred embodiment of the present disclosure relates to a pharmaceutical composition wherein the tonicity agent is sodium chloride present at a concentration of from about 5 mg/mL to about 12 mg/mL, from about 5.5 mg/mL to about 10 mg/mL, from about 6.0 mg/mL to about 10 mg/mL, from about 6.5 mg/mL to about 10 mg/mL, from about 7.0 mg/mL to about 10 mg/mL, from about 7.0 mg/mL to about 9 mg/mL, from about 7.0 mg/mL to about 8 mg/mL, about 7.72 mg/mL, about 8 mg/mL, or about 9 mg/mL of the composition.

The pharmaceutical compositions disclosed herein are contemplated to have a stable pH in pre-filled syringes over an extended period. Preferably, the pH drift is less than 2.0 pH units after storage over at least 2 months, preferably at least 6 months, more preferably at least 12 months, even more preferably at least 18 months, and most preferably at least 24 months, at 25° C. More preferably, the pH drift is less than 1.5 pH units after storage over at least 2 months, preferably at least 6 months, more preferably at least 12 months, more preferably at least 18 months, and most preferably at least 24 months, at 25° C. Even more preferably, the pH drift is less than 1.0 pH units after storage over at least 2 months, preferably at least 6 months, more preferably at least 12 months, even more preferably at least 18 months, and most preferably at least 24 months, at 25° C. Most preferably, the pH drift is less than 0.5 pH units after storage over at least 2 months, preferably at least 6 months, more preferably at least 12 months, even more preferably at least 18 months, and most preferably at least 24 months, at 25° C.

In some embodiments, the pH drift is less than 0.4 pH units after storage over at least 2 months, preferably at least 6 months, more preferably at least 12 months, even more preferably at least 18 months, and most preferably at least 24 months, at 25° C. In some embodiments, the pH drift is less than 0.3 pH units after storage over at least 2 months, preferably at least 6 months, more preferably at least 12 months, even more preferably at least 18 months, and most preferably at least 24 months, at 25° C. In some embodiments, the pH drift is less than 0.2 pH units after storage over at least 2 months, preferably at least 6 months, more preferably at least 12 months, even more preferably at least 18 months, and most preferably at least 24 months, at 25° C. In some embodiments, the pH drift is less than 0.1 pH units after storage over at least 2 months, preferably at least 6 months, more preferably at least 12 months, even more preferably at least 18 months, and most preferably at least 24 months, at 25° C.

In some embodiments, the pharmaceutical compositions disclosed herein exhibit a pH drift of less than 0.5 pH unit after storage for at least one week in pre-filled syringes under accelerated conditions of 60° C. In some embodiments, the pharmaceutical compositions disclosed herein exhibit a pH drift of less than 0.5 pH unit after storage for at least 2 weeks in pre-filled syringes under accelerated conditions of 60° C. In some embodiments, the pharmaceutical compositions disclosed herein exhibit a pH drift of less than 0.5 pH unit after storage for at least 3 weeks in pre-filled syringes under accelerated conditions of 60° C. In some embodiments, the pharmaceutical compositions disclosed herein exhibit a pH drift of less than 0.5 pH unit after storage for at least 4 weeks in pre-filled syringes under accelerated conditions of 60° C.

In certain embodiments, the pharmaceutical composition is a sterile aqueous injectable solution comprising about 1 mg/mL to about 10 mg/mL of bupivacaine hydrochloride, about 0.05 mg/mL to about 10 mg/mL of a buffer, about 5 mg/mL to about 15 mg/ml of a tonicity agent, and water, wherein the composition has an initial pH in the range of 4.0 to 6.5 and wherein the composition exhibits a pH drift of less than 0.5 pH unit.

In certain embodiments, the pharmaceutical composition is a sterile aqueous injectable solution comprising about 1 mg/mL to about 10 mg/mL of bupivacaine hydrochloride, about 0.05 mg/mL to about 10 mg/mL of a buffer, about 5 mg/mL to about 15 mg/mL of a tonicity agent, a pH adjuster in an amount sufficient to adjust the composition to an initial pH of from about 4.0 to about 6.5, and water, wherein the composition has a pH drift of less than 0.5 pH unit.

The pharmaceutical compositions according to the present disclosure may be sterilized. Sterilization can be achieved through conventional methods such as aseptic filling, sterile filtration, autoclaving, irradiation, or heat sterilization. In some embodiments, the composition is filter sterilized and then aseptically filled into sterilized syringes (which can be sterilized by X-ray or gamma irradiation, or by a gaseous sterilant, or autoclaving).

In addition, the inventor has also discovered that increasing the initial pH of an aqueous injectable composition containing bupivacaine hydrochloride to about 6.0 or about 6.5 ensures its stability in pre-filled syringes and effectively prevents or minimizes pH drift throughout the product's shelf life without requiring a buffer. By adjusting the initial pH to these levels, the aqueous injectable composition exhibits significantly improved stability in pre-filled syringes compared to currently available bupivacaine formulations and, more importantly, maintains stability for a commercially viable period. This enhanced stability facilitates the convenient distribution and sale of bupivacaine hydrochloride in pre-filled syringe formats.

Accordingly, certain embodiments of the present disclosure provide a pharmaceutical composition in a pre-filled syringe, comprising: bupivacaine hydrochloride, a tonicity agent, a pH adjuster in an amount sufficient to adjust the composition to an initial pH of about 6.0 or about 6.5, and water. In various embodiments, such a pharmaceutical composition comprises about 1 mg/mL to about 10 mg/mL of bupivacaine hydrochloride, about 5 mg/mL to about 15 mg/mL of a tonicity agent, a pH adjuster in an amount sufficient to adjust the composition to an initial pH of about 6.0 or about 6.5, and water, wherein the composition has a pH drift of less than 0.5 pH unit. As used herein, the term “initial adjusted pH” or “initial pH” refers to the pH level initially set during the preparation of the composition. After the composition is formulated and its pH is set to the initial value, it is expected to exhibit a pH drift of less than 0.5 pH units when stored in pre-filled syringes for at least two months, preferably at least six months of storage at room temperature, or for at least one week under accelerated storage conditions at 60° C.

The present disclosure also provides a pre-filled syringe (PFS) comprising a pharmaceutical composition according to the present disclosure. In one embodiment, the syringe is a plastic syringe, preferably a syringe made of polypropylene (PP), cyclic olefin copolymer (COC) and/or cyclic olefin polymer (COP). In certain preferred embodiments, the pre-filled syringe is made of cyclic olefin copolymer (COC). In one embodiment, the syringe has a total volume selected from 5 mL, 10 ml, 20 ml, and 50 ml.

In one embodiment, the pre-filled syringe is designed for single-dose administration.

In certain embodiments, the pre-filled syringe includes a syringe barrel, a plunger moveably positioned within the syringe barrel and a plunger rod for driving the plunger. The syringe barrel is made of cyclic olefin copolymer (COC), while the plunger is composed of rubber compound preferably made of chlorobutyl or bromobutyl.

The present disclosure further provides a method for improving stability of an injectable solution composition comprising about 1 mg/mL to about 10 mg/mL of bupivacaine hydrochloride, about 5 mg/mL to about 15 mg/mL of a tonicity agent, and water, with a pH ranging from about 4 to 6.5. The method involves incorporating a buffer into the composition such that the composition has a pH drift of less than 0.5 pH unit after storage for at least 2 months in a pre-filled syringe at 25° C., or after storage for at least one week in a pre-filled syringe under accelerated conditions of 60° C.

The present disclosure is also directed to a process for preparing the pharmaceutical composition disclosed herein. The process comprises the steps of: (i) dissolving bupivacaine hydrochloride, a buffer, and optionally a tonicity agent in water to form a solution, and (ii) adding a pH adjuster to adjust a pH range of the solution between about 4.0 and about 6.5 to provide the pharmaceutical composition. Preferably, the process further comprises a step of sterilizing the composition. The step of sterilizing may comprise sterile filtration, autoclaving, irradiation, or heat sterilization. The sterilized composition may be filled in a syringe to produce a pre-filled syringe according to the invention.

The present disclosure is also directed to a kit comprising a pre-filled syringe containing the pharmaceutical composition disclosed herein, and instructions for using the pharmaceutical composition.

While the foregoing description discloses various embodiments of the disclosure, other and further embodiments of the invention may be devised without departing from the basic scope of the disclosure. The invention is not limited to the described embodiments, versions, or examples, which are included to enable a person having ordinary skill in the art to make and use the invention when combined with information and knowledge available to the person having ordinary skill in the art.

### Examples

The present disclosure is further explained in the form of following examples. However, it is to be understood that the following examples are merely illustrative and are not to be taken as limitations upon the scope of the invention. Various changes and modifications to the disclosed embodiments will be apparent to those skilled in the art. Such changes and modifications may be made without departing from the scope of the invention. The appropriate procedure to prepare the injectable compositions of bupivacaine hydrochloride will be apparent to those skilled in the art. Unless indicated otherwise, pH was measured immediately after transferring the test solution from the indicated container after storage into a new container.

### Bupivacaine Hydrochloride Composition

Injectable solutions were prepared according to the ingredients and amounts presented in Tables 1 and 2. Examples 1 to 6 are illustrative of the present invention, while examples C1 to C3 are comparative examples. The compositions of examples C1 to C3 are equivalent to those of the currently available bupivacaine formulation marketed under the trade name Marcaine™.

TABLE 1

| | | Quantity mg/mL | | | | | |
|---|---|---|---|---|---|---|---|
| | | (buffered composition) | | | | | |
| Ingredients | Role | Ex. 1 | Ex. 2 | Ex. 3 | Ex. 4 | Ex. 5 | Ex. 6 |
| Bupivacaine HCl | Active | 2.5 | 5 | 7.5 | 2.5 | 5 | 7.5 |
| Sodium chloride | Tonicity | 7.72 | 7.47 | 7.22 | 7.72 | 7.47 | 7.22 |
| | Agent | | | | | | |
| Citric acid | Buffer | 0.2 | 0.2 | 0.2 | 0 | 0 | 0 |
| Sodium lactate | Buffer | 0 | 0 | 0 | 1.02 | 1.02 | 1.02 |
| Sodium hydroxide/ | pH | 4-6.5 | 4-6.5 | 4-6.5 | 4-6.5 | 4-6.5 | 4-6.5 |
| Hydrochloric acid | adjuster | (pH 5.5) | (pH 5.5) | (pH 5.5) | (pH 5.5) | (pH 5.5) | (pH 5.5) |
| Water for Injection | Vehicle | q.s. 1 mL | q.s. 1 mL | q.s. 1 mL | q.s. 1 mL | q.s. 1 mL | q.s. 1 mL |

TABLE 2

| | | Quantity mg/mL | | |
|---|---|---|---|---|
| | | (unbuffered composition) | | |
| Ingredients | Role | Ex. C1 | Ex. C2 | Ex. C3 |
| Bupivacaine HCl | Active | 2.5 | 5 | 7.5 |
| Sodium chloride | Tonicity Agent | 8.6 | 8.1 | 7.6 |
| Sodium hydroxide/ | pH adjuster | 4-6.5 | 4-6.5 | 4-6.5 |
| Hydrochloric acid | | (pH 5.5) | (pH 5.5) | (pH 5.5) |
| Water for Injection | Vehicle | q.s. 1 mL | q.s. 1 mL | q.s. 1 mL |

### Stability Studies

To evaluate the stability of the instantly disclosed compositions in pre-filled syringes compared to the marketed product Marcaine™ in pre-filled syringes, the inventors conducted a comparative study, the results of which are presented in Tables 3 to 6 below. The stability studies utilized the following container closure system (syringe).
10 ml Syringe
   Barrel: Schott™ COC with FM 257 Tip; X-Ray Sterilized
   Plunger: Datwyler™ FM 457/FM257; Gamma Sterilized
   Plunger Rod: Polypropylene
20 ML Syringe
   Barrel: Schott™ COC with FM 257 Tip; X-Ray Sterilized
   Plunger: Datwyler™ FM 457/FM 257; Gamma Sterilized
   Plunger Rod: Polypropylene

The data presented in Tables 3 to 6 indicate that while the currently available bupivacaine formulation (Marcaine™) remains stable in glass vials, it lacks pH stability when stored in pre-filled syringes made of polypropylene (PP) or cyclic olefin copolymer (COC). In contrast, the data demonstrate that the inventive compositions exhibit minimal pH drift and/or variability when stored in pre-filled syringes (particularly in the presence of buffer), compared to the currently available bupivacaine formulation.

TABLE 3
Accelerated Stability
(60° C. ± 2° C.)

| | | | | | | Buffered composition | |
|---|---|---|---|---|---|---|---|
| | | | | | | (2.5 mg/mL drug | |
| | | | | | | concentration) | |
| | Unbuffered composition | | | | | Ex. 1 | Ex. 4 |
| | (2.5 mg/mL drug concentration) | | | | | (Citric acid | (Lactic acid |
| | | | Ex. C1 in | | Ex. C1 in | buffered | buffered |
| | Ex. C1 in 10 | Ex. C1 in | 10 mL | Ex. C1 in | 10 mL | solution) in 10 | solution) in |
| | mL glass vial | 10 mL | glass | 10 mL PP | COC | mL COC | 10 mL COC |
| Time point | (Marcaine ™) | glass vial | syringe | syringe | syringe | syringe | syringe |
| Initial pH | 5.68 | 5.31 | 5.40 | 5.23 | 5.40 | 5.48 | 5.41 |
| pH after 1 | 5.58 | 5.91 | 4.71 | 4.20 | 5.08 | 5.33 | 5.34 |
| week | | | | | | | |
| pH after 2 | 5.49 | — | 4.55 | 4.12 | 4.79 | 5.40 | 5.41 |
| weeks | | | | | | | |
| pH after 3 | — | — | — | 3.98 | 4.75 | 5.37 | 5.32 |
| weeks | | | | | | | |
| pH after 4 | — | — | — | — | 4.95 | — | — |
| weeks | | | | | | | |

TABLE 4
Accelerated Stability
(60° C. ± 2° C.)

| | | | | | | Buffered composition | |
|---|---|---|---|---|---|---|---|
| | | | | | | (7.5 mg/mL drug | |
| | | | | | | concentration) | |
| | Unbuffered composition | | | | | Ex. 3 | Ex. 6 |
| | (7.5 mg/mL drug concentration) | | | | | (Citric acid | (Lactic acid |
| | | | Ex. C3 in | Ex. C3 in | Ex. C3 in | buffered | buffered |
| | Ex. C3 in 10 | Ex. C3 in | 10 mL | 10 mL | 10 mL | solution) in | solution) in |
| | mL glass vial | 10 mL | glass | PP | COC | 10 mL COC | 10 mL COC |
| Time point | (Marcaine ™) | glass vial | syringe | syringe | syringe | syringe | syringe |
| Initial pH | 5.68 | 5.31 | 5.47 | 5.30 | 5.42 | 5.53 | 5.54 |
| pH after 1 | 5.58 | 5.91 | 4.65 | 4.02 | 5.07 | 5.33 | 5.31 |
| week | | | | | | | |
| pH after 2 | 5.49 | 5.47 | 4.46 | 3.84 | 4.85 | 5.46 | 5.38 |
| weeks | | | | | | | |
| pH after 3 | — | 5.36 | — | 3.78 | 4.88 | 5.32 | 5.29 |
| weeks | | | | | | | |
| pH after 4 | — | 5.51 | — | — | 4.82 | — | — |
| weeks | | | | | | | |

TABLE 5
Accelerated Stability
(60° C. ± 2° C)

| | | | Buffered composition | |
|---|---|---|---|---|
| | Unbuffered composition | | (2.5 mg/mL drug concentration) | |
| | (2.5 mg/mL drug concentration) | | Ex. 1 | Ex. 4 |
| | Ex. C1 in | Ex. C1 in | (Citric acid buffered | (Lactic acid buffered |
| | 10 mL glass vial | 20 mL COC | solution) in 20 mL | solution) in 20 mL |
| Time point | (Marcaine ™) | syringe | COC syringe | COC syringe |
| Initial pH | 5.68 | 5.24 | 5.48 | 5.41 |
| pH after 1 week | 5.58 | 4.53 | 5.25 | 5.23 |
| pH after 2 weeks | 5.49 | 4.33 | 5.35 | 5.31 |
| pH after 3 weeks | — | 4.31 | 5.26 | 5.19 |
| pH after 4 weeks | — | 4.22 | — | — |

TABLE 6
Accelerated Stability
(60° C. ± 2° C)

| | | | Buffered composition | |
|---|---|---|---|---|
| | Unbuffered composition | | (7.5 mg/mL drug concentration) | |
| | (7.5 mg/mL drug concentration) | | Ex. 3 | Ex. 6 |
| | Ex. C3 in | Ex. C3 in | (Citric acid buffered | (Lactic acid buffered |
| | 10 mL glass vial | 20 mL COC | solution) in 20 mL | solution) in 20 mL |
| Time point | (Marcaine ™) | syringe | COC syringe | COC syringe |
| Initial pH | 5.68 | 5.40 | 5.53 | 5.54 |
| pH after 1 week | 5.58 | 4.62 | 5.26 | 5.22 |
| pH after 2 weeks | 5.49 | 4.35 | 5.33 | 5.27 |
| pH after 3 weeks | — | 4.28 | 5.18 | 5.12 |
| pH after 4 weeks | — | 4.17 | — | — |

Additional experiments were performed with formulations as prepared in Tables 7A-7C. More particularly, Table 7A shows the composition for unbuffered bupivacaine-containing solutions at lower (0.25%), medium (0.5%), and higher (0.75%) concentrations, each at pH 5.5. Table 7B shows the composition for unbuffered bupivacaine-containing solutions at lower (0.25%) concentration at pH 6.0. Table 7C shows the composition for lactate-buffered bupivacaine-containing solutions at lower (0.25%) and medium (0.5%) concentrations, each at pH 5.5. The stability data for these formulations are presented in the data of Tables 8A-12D for storage conditions as indicated in the tables.

TABLE 7A
NVK048 (Bupivacaine Hydrochloride Injection)
Unbuffered composition at pH 5.5 (lot 16745)

| Ingredient | 0.25% | 0.5% | | 0.75% |
|---|---|---|---|---|
| Bupivacaine | 2.5 mg/mL | 5 | mg/ml | 7.5 mg/mL |
| Hydrochloride | | | | |
| Sodium Chloride | 8.6 mg/mL | 8.1 | mg/mL | 7.6 mg/mL |

| | | | |
|---|---|---|---|
| Sodium Hydroxide | q.s. pH 5.5 | q.s. pH 5.5 | q.s. pH 5.5 |
| Hydrochloric Acid | | | |
| Water for Injection | q.s. 1 mL | q.s. 1 mL | q.s. 1 mL |

TABLE 7B
NVK048 (Bupivacaine Hydrochloride Injection)
Unbuffered composition at pH 6.0 (lot 16690)

| Ingredient | 0.25% |
|---|---|
| Bupivacaine Hydrochloride | 2.5 mg/mL |
| Sodium Chloride | 8.6 mg/mL |
| 1N Sodium Hydroxide | q.s. pH 6.0 |
| 1N Hydrochloric Acid | (pH 4.0-6.5) |
| Water for Injection | q.s. 1 mL |

TABLE 7C
NVK048 (Bupivacaine Hydrochloride Injection)
Buffered composition at pH 5.5 (lots 16897, 16930, 17194)

| Ingredient | 0.25% | | 0.5% | |
|---|---|---|---|---|
| Bupivacaine | 2.5 | mg/mL | 5 | mg/mL |
| Hydrochloride | | | | |
| Sodium Chloride | 7.72 | mg/mL | 7.47 | mg/mL |

| | | |
|---|---|---|
| Sodium DL-Lactate | 0.0017 mL/mL | 0.0017 mL/mL |
| Solution 60% | (equivalent | (equivalent |
| | to1.02 mg/mL) | to1.02 mg/mL) |
| 1N Sodium Hydroxide | q.s. pH 5.5 | q.s. pH 5.5 |
| 1N Hydrochloric Acid | (pH 4.0-6.5) | (pH 4.0-6.5) |
| Water for Injection | q.s. 1 mL | q.s. 1 mL |

Table 8A shows the results for Stability of Bupivacaine Hydrochloride-pH 5.5 (0.25% unbuffered) in 10 mL cyclo-olefin copolymer syringes (Lot #16745), and Table 8B shows the results for Stability of Bupivacaine Hydrochloride (0.25% unbuffered) in 20 mL cyclo-olefin copolymer syringes (Lot #16745). Here, CCS denotes clear colorless solution; NMT denotes not more than; and * denotes pH measurements were taken for individual syringes.

TABLE 8A

| | | Storage Conditions | | | | | |
|---|---|---|---|---|---|---|---|
| | | 40° C./NMT 25% RH | | 25° C./40% RH | | | |
| | | Time Point | | | | | |
| | Initial | 3 Month | 6 Month* | 3 Month | 6 Month* | 9 Month* | 12 Month* |
| Appearance | CCS | CCS | CCS | CCS | CCS | CCS | CCS |
| pH | 5.31 | 5.23 | 5.38 | 5.52 | 5.71 | 5.67 | 5.43, |
| | | | 5.15 | | 5.61 | 5.61 | 5.50 |
| | | | 5.14 | | 5.67 | | |
| | | | | | 5.63 | | |
| Assay of Bupivacaine | 100.7 | 101.1 | 100.0 | 100.0 | 99.9 | 101.0 | 100.9 |
| HCl (%) | | | | | | | |

TABLE 8B

| | | Storage Conditions | | | | |
|---|---|---|---|---|---|---|
| | | 40° C./NMT 25% RH | | 25° C./40% RH | | |
| | | Time Point | | | | |
| | Initial | 3 Month | 6 Month* | 3 Month* | 6 Month* | 12 Month* |
| Appearance | CCS | CCS | CCS | CCS | CCS | CCS |
| pH | 5.31 | 4.69 | 4.46, | 5.24, | 5.08 | 4.96 |
| | | | 5.13 | 5.15 | 5.10 | 4.88 |
| | | | | | 5.10 | |
| | | | | | 5.06 | |
| Assay of Bupivacaine | 100.5 | 101.3 | 99.8 | 100.6 | 100.0 | 101.1 |
| HCl (%) | | | | | | |

Table 9A shows results for stability of Bupivacaine Hydrochloride pH 5.5 (0.5% unbuffered) in 10 mL cyclo-olefin copolymer syringes (Lot #16745), and Table 9B shows results for stability of Bupivacaine Hydrochloride (0.5% unbuffered) in 20 mL cyclo-olefin copolymer syringes (Lot #16745). Here, CCS denotes clear colorless solution; NMT denotes not more than; and * denotes pH measurements were taken for individual syringes.

TABLE 9A

| | | Storage Conditions | | | | | |
|---|---|---|---|---|---|---|---|
| | | 40° C./NMT 25% RH | | 25° C./40% RH | | | |
| | | Time Point | | | | | |
| | Initial | 3 Month | 6 Month* | 3 Month | 6 Month* | 9 Month* | 12 Month* |
| Appearance | CCS | CCS | CCS | CCS | CCS | CCS | CCS |
| pH | 5.41 | 5.28 | 5.04, | 5.50 | 5.53, | 5.53, | 5.51, |
| | | | 5.08, | | 5.65, | 5.58 | 5.47 |
| | | | 4.99, | | 5.56, | | |
| | | | 4.98 | | 5.64 | | |
| Assay of Bupivacaine | 101.1 | 101.0 | 99.2 | 99.3 | 98.4 | 100.3 | 100.9 |
| HCl (%) | | | | | | | |

TABLE 9B

| | | Storage Conditions | | | | | |
|---|---|---|---|---|---|---|---|
| | | 40° C./NMT 25% RH | | 25° C./40% RH | | | |
| | | Time Point | | | | | |
| | Initial | 3 Month | 6 Month* | 3 Month* | 6 Month* | 9 Month* | 12 Month* |
| Appearance | CCS | CCS | CCS | CCS | CCS | CCS | CCS |
| pH | 5.33 | 4.66 | 4.43, | 5.26, | 5.01, | 4.95, | 4.93, |
| | | | 4.42 | 5.19 | 5.05, | 4.93 | 4.88 |
| | | | | | 5.06, | | |
| | | | | | 5.11 | | |
| Assay of Bupivacaine | 100.0 | 100.7 | 99.8 | 100.8 | 99.3 | 101.2 | 100.3 |
| HCl (%) | | | | | | | |

Table 10A shows the results for stability of Bupivacaine Hydrochloride (0.75% unbuffered) in 10 mL cyclo-olefin copolymer syringes (Lot #16745), and Table 10B shows the results for stability of Bupivacaine Hydrochloride (0.75% unbuffered) in 20 mL cyclo-olefin copolymer syringes (Lot #16745). Here, CCS denotes clear colorless solution; NMT denotes not more than; and * denotes pH measurements were taken for individual syringes.

TABLE 10A

| | | Storage Conditions | | | | | |
|---|---|---|---|---|---|---|---|
| | | 40° C./NMT 25% RH | | 25° C./40% RH | | | |
| | | Time Point | | | | | |
| | Initial | 3 Month | 6 Month* | 3 Month | 6 Month* | 9 Month* | 12 Month* |
| Appearance | CCS | CCS | CCS | CCS | CCS | CCS | CCS |
| pH | 5.38 | 5.24 | 5.07 | 5.23 | 5.36 | 5.45 | 5.43 |
| | | | 4.98 | | 5.41 | 5.45 | 5.42 |
| | | | 5.13 | | 5.43 | | |
| | | | 5.03 | | 5.42 | | |
| Assay of Bupivacaine | 100.2 | 101.5 | 101.2 | 101.1 | 99.9 | 101.5 | 100.9 |
| HCl (%) | | | | | | | |

TABLE 10B

| | | Storage Conditions | | | | |
|---|---|---|---|---|---|---|
| | | 40° C./NMT 25% RH | | 25° C./40% RH | | |
| | | Time Point | | | | |
| | Initial | 3 Month | 6 Month* | 3 Month* | 6 Month* | 9 Month* |
| Appearance | CCS | CCS | CCS | CCS | CCS | CCS |
| pH | 5.28 | 4.64 | 4.38, | 5.26, | 5.04, | 4.93, |
| | | | 4.39 | 5.15 | 4.98, | 4.92 |
| | | | | | 5.02, | |
| | | | | | 4.99 | |
| Assay of Bupivacaine | 100.3 | 101.5 | 100.1 | 100.9 | 100.3 | 100.9 |
| HCl (%) | | | | | | |

Table 11 shows the results for stability of Bupivacaine Hydrochloride (0.25% unbuffered) in 10 mL cyclo-olefin copolymer syringes (Lot #16690), where CCS denotes clear colorless solution; NMT denotes not more than; and * denotes pH measurements were taken for individual syringes.

TABLE 11

| | | Storage Conditions | | | | | |
|---|---|---|---|---|---|---|---|
| | | 40° C./NMT 25% RH | | 25° C./40% RH | | | |
| | | Time Point | | | | | |
| | Initial | 3 Month | 6 Month | 3 Month | 6 Month | 9 Month | 12 Month |
| Appearance | CCS | CCS | CCS | CCS | CCS | CCS | CCS |
| pH | 6.41 | 4.96 | 4.64 | 5.73 | 5.54 | 5.32 | 5.35 |
| Osmolality (mOsm/kg) | 291 | 291 | 291 | 293 | 292 | 291 | 292 |
| Assay of Bupivacaine HCl | 100.1 | 100.7 | 100.3 | 100.5 | 100.2 | 100.3 | 100.9 |
| (%) | | | | | | | |

Table 12A shows the results for stability of Bupivacaine Hydrochloride (0.25% buffered) in 10 mL cyclo-olefin copolymer syringes (Lot #17194), Table 12B shows the results for stability of Bupivacaine Hydrochloride (0.25% buffered) in 20 mL cyclo-olefin copolymer syringes (Lot #16897), Table 12C shows the results for stability of Bupivacaine Hydrochloride (0.5% buffered) in 10 mL cyclo-olefin copolymer syringes (Lot #17194), and Table 12D shows results for stability of Bupivacaine Hydrochloride-pH 5.5 (0.5% buffered) in 20 mL cyclo-olefin copolymer syringes (Lot #16930). Here, CCS denotes clear colorless solution; and * denotes pH measurements were taken for individual syringes.

TABLE 12A

| | | Storage Conditions | | | 25° C./ |
|---|---|---|---|---|---|
| | | 40° C./NMT 25% RH | | | 40% RH |
| Time Point | Initial | 1 Month | 2 Month | 3 Month | 3 Month |
| Appearance | CCS | CCS | CCS | CCS | CCS |
| PH | 5.42 | 5.38 | 5.37 | 5.36 | 5.45 |
| Assay of | 99.9 | 99.9 | 99.5 | 99.9 | 100.2 |
| Bupivacaine | | | | | |
| HCl (%) | | | | | |

TABLE 12B

| | | Storage Conditions | | | | | | |
|---|---|---|---|---|---|---|---|---|
| | | 40° C./NMT 25% RH | | | | 25° C./40% RH | | |
| | | Time Point | | | | | | |
| | Initial | 1 Month | 2 Month | 3 Month | 6 Month | 3 Month | 6 Month | 9 Month |
| Appearance | CCS | CCS | CCS | CCS | CCS | CCS | CCS | CCS |
| pH | 5.49 | 5.54 | 5.29 | 5.32 | 5.38 | 5.42 | 5.50 | 5.43 |
| Assay of Bupivacaine HCl (%) | 100.5 | 100.0 | 99.8 | 100.2 | 100.2 | 101.6 | 100.0 | 100.3 |

TABLE 12C

| | | Storage Conditions | | | 25° C./ |
|---|---|---|---|---|---|
| | | 40° C./NMT 25% RH | | | 40% RH |
| Time Point | Initial | 1 Month | 2 Month | 3 Month | 3 Month |
| Appearance | CCS | CCS | CCS | CCS | CCS |
| pH | 5.44 | 5.38 | 5.31 | 5.29 | 5.47 |
| Assay of | 100.7 | 100.1 | 100.2 | 99.6 | 99.8 |
| Bupivacaine | | | | | |
| HCl (%) | | | | | |

TABLE 12D

| | | Storage Conditions | | | | | | |
|---|---|---|---|---|---|---|---|---|
| | | 40° C./NMT 25% RH | | | | 25° C./40% RH | | |
| | | Time Point | | | | | | |
| | Initial | 1 Month | 2 Month | 3 Month | 6 Month | 3 Month | 6 Month | 9 Month |
| Appearance | CCS | CCS | CCS | CCS | CCS | CCS | CCS | CCS |
| pH | 5.48 | 5.40 | 5.26 | 5.19 | 5.12 | 5.31 | 5.31 | 5.43 |
| Assay of Bupivacaine HCl (%) | 99.4 | 102.1 | 98.4 | 100.9 | 98.9 | 101.0 | 100.6 | 99.7 |

Notably, and as can be taken from the above, pH stability was typically better at smaller volumes (e.g., 10 mL compared to 20 mL), and variability of the observed pH values in the 10 mL syringes was reduced by addition of the buffer, which was more pronounced in the 20 mL syringes.

Having now fully described this invention, it will be understood by those of ordinary skill in the art that it can be performed within a wide equivalent range of parameters without affecting the scope of the invention or any embodiment thereof. All publications, patent applications and patents disclosed herein are incorporated by reference in their entirety.

1. A pharmaceutical composition in a pre-filled syringe, comprising:
bupivacaine hydrochloride in water;
a tonicity agent;
a buffer;
a pH adjuster in an amount sufficient to adjust the composition to an initial pH of from about 4.0 to about 6.5; and
wherein the composition has a pH drift of less than 0.5 pH unit after storage for at least 2 months at 25° C.; or
wherein the composition has a pH drift of less than 0.5 pH unit after storage for at least 6 months at 25° C.; or
wherein the composition has a pH drift of less than 0.5 pH unit after storage for at least 12 months at 25° C.

2. The pharmaceutical composition of claim 1, wherein the initial adjusted pH of the composition is from about 4.0 to about 5.6.

3. The pharmaceutical composition of claim 1, wherein the initial adjusted pH of the composition is from about 5.4 to about 6.5.

4. The pharmaceutical composition of claim 1, wherein the initial adjusted pH of the composition is about 5.5.

5. The pharmaceutical composition of claim 1,
wherein the composition has a pH drift of less than 0.5 pH unit after storage for at least 2 months at 25° C.; and
wherein the composition has a pH drift of less than 0.5 pH unit after storage for at least 6 months at 25° C.; and
wherein the composition has a pH drift of less than 0.5 pH unit after storage for at least 12 months at 25° C.

6. The pharmaceutical composition of claim 1,
wherein the composition has a pH drift of less than 0.5 pH unit after storage for at least 4 weeks at 60° C.

7. The pharmaceutical composition of claim 1, wherein the bupivacaine hydrochloride is present at a concentration of from about 1 mg/mL to about 10 mg/mL.

8. The pharmaceutical composition of claim 1, wherein the bupivacaine hydrochloride is present at a concentration of about 2.5 mg/mL, about 5 mg/mL, or about 7.5 mg/mL.

9. The pharmaceutical composition of claim 1, wherein the buffer is selected from citric acid, sodium citrate, lactic acid, sodium lactate, succinic acid, sodium succinate, sodium acetate, acetic acid, sodium phosphate, phosphoric acid, sodium ascorbate, tartaric acid, maleic acid, ascorbic acid, sodium bicarbonate, histidine, sodium benzoate, benzoic acid, and combinations thereof.

10. The pharmaceutical composition of claim 9, wherein the buffer is citric acid.

11. The pharmaceutical composition of claim 9, wherein the buffer is lactic acid.

12. The pharmaceutical composition of claim 1, wherein the buffer is present at a concentration of from about 0.05 mg/mL to about 10 mg/mL.

13. The pharmaceutical composition of claim 12, wherein the buffer is present at a concentration of from about 0.1 mg/mL to about 5 mg/mL.

14. The pharmaceutical composition of claim 1, wherein the pH adjuster is selected from sodium hydroxide, hydrochloric acid, and a combination thereof.

15. The pharmaceutical composition of claim 1, wherein the water is water for injection (WFI).

16. The pharmaceutical composition of claim 1, wherein the tonicity agent is selected from a group consisting of sugar (such as dextrose), glycerin, a sugar alcohol (such as mannitol), sodium chloride, and combinations thereof.

17. The pharmaceutical composition of claim 1, wherein the tonicity agent is sodium chloride.

18. The pharmaceutical composition of claim 1, wherein the tonicity agent is present at a concentration of from about 5 mg/mL to about 15 mg/mL.

19. The pharmaceutical composition of claim 1, wherein the composition comprises:
about 1 mg/mL to about 10 mg/mL of bupivacaine hydrochloride;
about 0.05 mg/mL to about 10 mg/mL of a buffer;
about 5 mg/mL to about 15 mg/mL of a tonicity agent;
a pH adjuster in an amount sufficient to adjust the composition to an initial pH of from about 4.0 to about 6.5; and
water, wherein the composition has a pH drift of less than 0.5 pH unit.

20. The pharmaceutical composition of claim 1, wherein the composition is sterilized.

21. The pharmaceutical composition of claim 20, wherein the composition is sterilized using aseptic filling, sterile filtration, irradiation, or heat sterilization.

22. A pre-filled syringe comprising the pharmaceutical composition of claim 1.

23. The pharmaceutical composition of claim 1, wherein the pre-filled syringe comprises a syringe barrel, a plunger moveably positioned within the syringe barrel and a plunger rod for driving the plunger.

24. The pharmaceutical composition of claim 23, wherein the syringe barrel is made of cyclic olefin copolymer (COC).

25. The pharmaceutical composition of claim 23, wherein the plunger is made of chlorobutyl or bromobutyl.

26. The pharmaceutical composition of claim 1, wherein the pre-filled syringe has a total volume selected from 5 mL, 10 ml, 20 ml, and 50 ml.

27. A method of improving stability of an injectable solution composition comprising:
about 1 mg/mL to about 10 mg/mL of bupivacaine hydrochloride;
about 5 mg/mL to about 15 mg/mL of a tonicity agent; and
water, and having a pH of about 4.0 to about 6.5,
said method comprising the step of incorporating a buffer into the composition such that the composition has a pH drift of less than 0.5 pH unit after storage for at least 2 months in a pre-filled syringe at 25°, or after storage for at least one week in a pre-filled syringe under accelerated conditions of 60° C.
